(19) United States
(12) Patent Application Publication
(10) Pub. No.: US 2026/0014360 A1
(43) Pub. Date: Jan. 15, 2026

(54) MICRONEEDLE DEVICE AND MEDICINE ADMINISTRATION/REACTION PRODUCT WITHDRAWAL METHOD

(71) Applicant: Murata Manufacturing Co., Ltd., Nagaokakyo-shi (JP)

(72) Inventors: Hideyuki KUMITA, Nagaokakyo-shi (JP); Daisho TSUBOKAWA, Nagaokakyo-shi (JP); Ryota MURAKAMI, Nagaokakyo-shi (JP); Shinichi OIDE, Nagaokakyo-shi (JP); Hiroyuki HARANO, Nagaokakyo-shi (JP)

(21) Appl. No.: 19/334,067

(22) Filed: Sep. 19, 2025

Related U.S. Application Data
(63) Continuation of application No. PCT/JP2023/043642, filed on Dec. 6, 2023.

(30) Foreign Application Priority Data
Mar. 30, 2023 (JP) 2023-055948

(51) Int. Cl. A61M 37/00

(52) U.S. Cl. CPC A61M 37/0015; A61M 2037/0023; A61M 2037/0061

(57) ABSTRACT

A microneedle device including: a substrate; and microneedles on an attachment surface on a surface of the substrate, in which a medicine is subcutaneously administered by attaching the attachment surface including the microneedles to a living body, and a reaction product generated by the subcutaneous administration of the medicine is withdrawn.

### CROSS REFERENCE TO RELATED APPLICATIONS

The present application is a continuation of International application No. PCT/JP2023/043642, filed Dec. 6, 2023, which claims priority to Japanese Patent Application No. 2023-055948, filed Mar. 30, 2023, the entire contents of each of which are incorporated herein by reference.

### TECHNICAL FIELD

The present disclosure relates to a microneedle device and a medicine administration/reaction product withdrawal method.

### BACKGROUND ART

Patent Document 1 describes a transdermal drug administration device with microneedles including a plurality of microneedles capable of perforating a skin.

According to Patent Document 1, by perforating the skin (stratum corneum) using microneedles at the time of transdermal administration of a physiologically active substance, a therapeutic effect by transdermal administration (passive diffusion) or iontophoresis administration of a physiologically active substance can be enhanced.
Patent Document 1: WO 2007/091608 A

### SUMMARY OF THE DISCLOSURE

The device used in Patent Document 1 includes a solution reservoir containing a solution for dissolving a drug, and the solution reservoir is opened by pressing the solution reservoir to supply the solution to dissolve a dry drug arranged in microneedles or the like. Then, the solution is supplied and a skin is perforated with the microneedles, and the drug dissolved in the solution is transdermally administered through the microneedles.

Some devices using a microneedle aim to sense information in a body by perforating the skin with the microneedle. In that case, the device needs to have a function of withdrawing a substance present in the body or measuring a physical property value in the body in order to obtain information in the body.

Patent Document 1 focuses on “administration” in which a drug is transdermally administered via microneedles, but does not consider a function of withdrawing a substance present in the body.

The present inventors have considered a device in which a medicine is subcutaneously administered using microneedles and a reaction product generated by a reaction of the medicine in the body is withdrawn. In this case, since the reaction product generated by the administration of the medicine is withdrawn, it is necessary to administer the medicine first and then withdraw the reaction product. That is, a required function is different from that of a device on the premise that administration of a medicine and withdrawal of a substance present in the body are performed simultaneously.

The present disclosure has been made in view of the above current situation, and an object thereof is to provide a device capable of subcutaneously administering a medicine and withdrawing a reaction product generated by a reaction of the medicine in the body with a time difference.

A microneedle device of the present disclosure includes: a substrate; and microneedles on an attachment surface on a surface of the substrate, the microneedle device being constructed to subcutaneously administer a medicine by attaching the attachment surface including the microneedles to a living body, and to withdraw a reaction product generated by the subcutaneous administration of the medicine.

The medicine administration/reaction product withdrawal method of the present disclosure includes: preparing a microneedle device including a substrate and microneedles on an attachment surface on a surface of the substrate; subcutaneously administering a medicine by attaching the attachment surface including the microneedles to a living body; and withdrawing a reaction product generated by the subcutaneous administration of the medicine.

According to the present disclosure, it is possible to provide a device capable of subcutaneously administering a medicine and withdrawing a reaction product generated by a reaction of the medicine in the body with a time difference.

### BRIEF EXPLANATION OF THE DRAWINGS

FIG. 1 is a view illustrating an example of a microneedle device of the present disclosure at the time of use.

FIG. 2 is a schematic sectional view of a lipolysis device and skin tissue when the lipolysis device is used.

FIG. 3 is a sectional view schematically illustrating an example of an embodiment of a microneedle device.

FIG. 4 is a sectional view schematically illustrating an example of the embodiment of a microneedle device.

FIG. 5 is a sectional view schematically illustrating an example of the embodiment of a microneedle device.

FIG. 6 is a sectional view schematically illustrating an example of the embodiment of a microneedle device.

FIG. 7 is a sectional view schematically illustrating an example of a procedure for performing administration of a medicine and withdrawal of a reaction product in a fourth embodiment of a microneedle device.

FIG. 8 is a sectional view schematically illustrating an example of a procedure for performing administration of a medicine and withdrawal of a reaction product in the fourth embodiment of the microneedle device.

FIG. 9 is a sectional view schematically illustrating an example of a procedure for performing administration of a medicine and withdrawal of a reaction product in the fourth embodiment of the microneedle device.

FIG. 10 is a perspective view schematically illustrating an example of a procedure for performing administration of a medicine and withdrawal of a reaction product in a fifth embodiment of a microneedle device.

FIG. 11 is a perspective view schematically illustrating an example of a procedure for performing administration of a medicine and withdrawal of a reaction product in the fifth embodiment of the microneedle device.

FIG. 12 is a perspective view schematically illustrating an example of a procedure for performing administration of a medicine and withdrawal of a reaction product in the fifth embodiment of the microneedle device.

FIG. 13 is a sectional view schematically illustrating an example of the embodiment of a microneedle device.

FIG. 14 is a sectional view schematically illustrating an example of the embodiment of a microneedle device.

FIG. 15 is a perspective view schematically illustrating an example of the embodiment of a microneedle device.

FIG. 16 is a graph schematically showing a concentration distribution of a reaction product along an axis indicated by line X-X in FIG. 15.

### DESCRIPTION OF THE PREFERRED EMBODIMENTS

Hereinafter, the microneedle device of the present disclosure and the medicine administration/reaction product withdrawal method will be described.

However, the present disclosure is not limited to a configuration below, and can be appropriately modified and applied within a range in which the spirit of the present disclosure is not changed. It should be noted that a combination of two or more of the individual preferred configurations of the present disclosure described below is also the present disclosure.

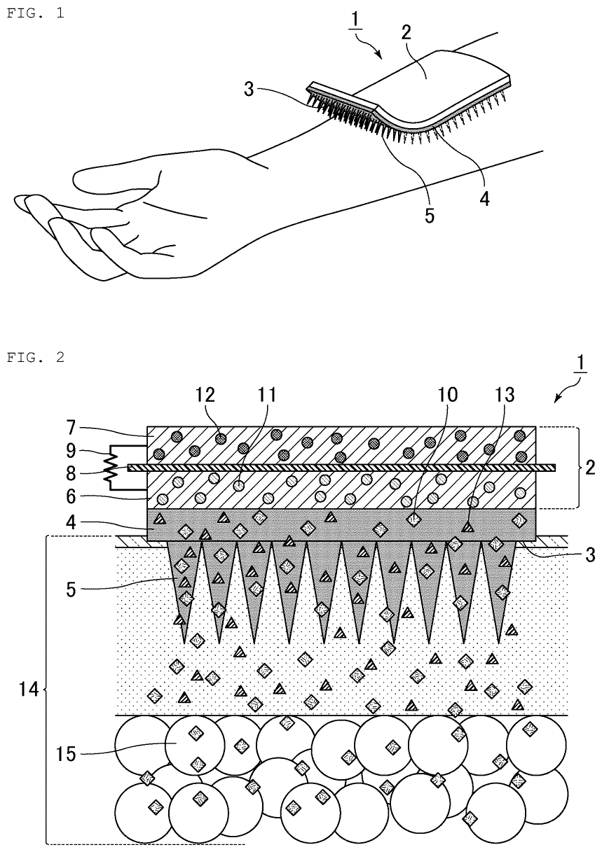

FIG. 1 is a view illustrating an example of the microneedle device of the present disclosure at the time of use.

A microneedle device 1 includes microneedles 5 provided on an attachment surface 3 of a surface of a substrate 4. A user presses the microneedles 5 against a skin of the user, whereby the microneedles 5 are inserted into the skin.

The surface of the microneedle device 1 opposite to the surface of the microneedles 5 is provided with a reaction product use portion 2 which is a device that operates using the withdrawn reaction product. As the device of the reaction product use portion 2, a battery unit can be exemplified.

Hereinafter, as a use example of the microneedle device of the present disclosure, an example of a lipolysis device in which a lipolytic enzyme as a medicine is subcutaneously administered, glycerol generated by decomposing fat by the lipolytic enzyme is withdrawn as a reaction product, and power generation is performed using the withdrawn glycerol will be described.

FIG. 2 is a schematic sectional view of a lipolysis device and skin tissue when the lipolysis device is used.

A microneedle device 1 that is a lipolysis device includes a substrate 4 and microneedles 5 provided on an attachment surface 3 of the substrate 4. A reaction product use portion 2, which is a battery unit, is provided on the surface of the substrate 4 opposite to the surface of the microneedles 5. In the battery unit, an anode (glycerol oxidation portion) 6 is arranged so as to be in contact with the substrate 4, a separator 8 and a cathode 7 are laminated thereon in this order, and the anode 6 and the cathode 7 are connected by an external circuit 9. The microneedles 5 are inserted into the skin, and a lipolytic enzyme 10 diffuses into skin tissue 14 from the microneedles 5. The lipolytic enzyme 10 reaches adipocytes 15 by the diffusion and decomposes fat. Glycerol 13 generated by the decomposition of fat is oxidized by glycerol oxidase 11 in the anode 6 when reaching the anode 6 through the microneedles 5. As a result, electrons and protons are obtained, and these electrons and protons move to the cathode 7 via the external circuit 9 and the separator 8, respectively, and oxygen is reduced by oxygen reductase 12 in the cathode 7, thereby generating electric energy, and providing a function as a battery.

In the microneedle device of the present disclosure, as in the lipolysis device of the above example, a medicine (lipolytic enzyme) is subcutaneously administered, and a reaction product (glycerol) generated by a reaction of the medicine in a body is withdrawn. Subcutaneous administration of a medicine is performed first, and withdrawal of a reaction product is performed later.

That is, the microneedle device of the present disclosure is a device that subcutaneously administers a medicine and withdraws a reaction product with a time difference.

Hereinafter, a general configuration of the microneedle device of the present disclosure will be described.

Unless otherwise mentioned in the description of each embodiment of the microneedle device of the present disclosure, the following general configurations of a medicine, a reaction product, a microneedle, and a substrate can be commonly applied.

### [General Configurations of Medicine and Reaction Product]

Examples of the medicine include lipolytic enzymes, activators of lipolytic enzymes, and substances indirectly promoting lipolysis.

Examples of the reaction product include glycerol and fatty acids.

It is preferable that the medicine is a lipolytic enzyme or a peptide compound having an activity equivalent thereto, and the reaction product is glycerol.

The lipolytic enzyme or a peptide compound having an activity equivalent thereto is not particularly limited as long as the lipolytic enzyme or peptide compound can decompose fat into glycerol and fatty acids. Specific examples of the lipolytic enzyme include lipase and analogues of lipase. The enzyme activity of the peptide compound is preferably from 50 to 120 inclusive when the enzyme activity of lipase is 100. A preferable example of the lipolytic enzyme or the like is lipase or analogues of lipase.

Analogues of the lipolytic enzyme are not particularly limited as long as the analogues of the lipolytic enzyme can decompose fat into glycerol and fatty acids. Examples of the analogues include chemically modified lipase. Examples of the chemically modified lipase include lipase bound with, for example, cell membrane permeable peptide or the like.

An average molecular weight of the lipolytic enzyme or the like is preferably from 2,000 to 100,000 inclusive. This improves permeability of the lipolytic enzyme or the like to adipocytes. The average molecular weight is preferably 5,000 to 80,000 inclusive, and further preferably from 10,000 to 70,000 inclusive.

The average molecular weight of the lipolytic enzyme or the like can be measured by ultracentrifugal analysis.

Examples of the activator of the lipolytic enzyme include a factor that activates lipase.

Examples of the substance indirectly promoting lipolysis include adrenaline and noradrenaline.

### [General Configuration of Microneedle]

The microneedle is preferably a porous body and/or a hollow body. The microneedle is more preferably a porous body. When the microneedle is a porous body, the reaction product can be withdrawn by capillary force of the porous body in addition to diffusion based on the concentration gradient of the reaction product in body fluids, so that the introduction efficiency of the reaction product is improved. Also, when the medicine is adsorbed to the microneedle, adsorption of the medicine to the microneedle is also improved since the microneedle is a porous body.

When the microneedle is a porous body, D50 in the pore diameter distribution is preferably 0.05 μm to 10 μm. This further improves the withdrawal efficiency of the reaction product. D50 in the pore diameter distribution is more preferably 0.5 μm to 5 μm. D50 in the pore diameter distribution can be obtained from a pore diameter distribution curve measured by a mercury intrusion method [horizontal axis: pore diameter (μm), vertical axis: log differential pore volume (mL/g)].

The microneedle preferably has a hydrophilic surface. When the surface of the microneedle has high hydrophilicity, an affinity with a highly hydrophilic reaction product such as glycerol is further enhanced, and the withdrawal efficiency of the hydrophilic reaction product is further improved.

In addition, when the microneedle is a porous body and the surface of the microneedle has high hydrophilicity, it is possible to selectively withdraw a highly hydrophilic reaction product such as glycerol more than a hydrophobic reaction product such as a fatty acid due to the capillary force of the porous body in addition to diffusion based on the concentration gradient of the reaction product in body fluids, and thus the withdrawal efficiency of the hydrophilic reaction product is further improved.

Examples of the method for hydrophilizing the surface of the microneedle include a method of imparting a hydroxyl group to the surface of the microneedle using plasma or the like.

The contact angle of the surface of the microneedle with water is preferably 60° or less. In this case, hydrophilicity of the microneedle becomes more sufficient. The contact angle is more preferably 50° or less, further preferably 40° or less, and particularly preferably 30° or less.

The contact angle can be measured by a contact angle meter.

A shape of the microneedle is not particularly limited as long as the microneedle having the shape can pierce a skin, but the shape is preferably conical or pyramidal. More preferably, the shape is conical.

The material of the microneedle is preferably a polymer, metal, resin, or the like that does not harm a living body and is compatible with the living body. Specific examples of the material of the microneedle include alginate, hyaluronic acid, curdlan, chitin, chitosan, glucomannan, polymalic acid, collagen, collagen peptide, hydroxypropyl cellulose, gelatin, silicon, titanium, silicone, polylactic acid (PLA), polyglycolic acid (PGA), and PLA-PGA copolymers, and plasma-treated materials of these. Among these, biodegradable materials are more preferable, hyaluronic acid, collagen, and polylactic acid are further preferable, and polylactic acid is especially preferable.

A length of the microneedle is not particularly limited as long as the microneedle having the length can pierce a skin, but the length is preferably from 100 μm to 3,000 μm inclusive. This allows the medicine to be more sufficiently diffused into subcutis in a less invasive manner. The length of the microneedle is more preferably from 150 μm to 1,500 μm inclusive, further preferably from 150 μm to 1,000 μm inclusive, and especially preferably from 200 μm to 800 μm inclusive.

A diameter of a bottom of the microneedle (the maximum diameter of the microneedle) is preferably from 50 μm to 1,000 μm inclusive. This makes it possible for the microneedle to pierce a skin in a less invasive manner. The diameter of the bottom of the microneedle is more preferably from 100 μm to 800 μm inclusive.

The number of microneedles included in the microneedle device is not particularly limited, but the number is preferably from 25 to 250,000 inclusive. More preferably, the number is from 2,500 to 50,000 inclusive.

The density of microneedles is not particularly limited, but the density is preferably from 1/cm² to 10,000/cm² inclusive. More preferably, the density is from 100/cm² to 2,000/cm² inclusive.

The method for manufacturing microneedles is not particularly limited, and examples of the method include a method for injection-molding a material for forming the microneedles.

### [General Configuration of Substrate]

The substrate is not particularly limited as long as it supports the microneedles, and may be made of the same material as or a different material from that of the microneedles, but the same material is preferable.

The thickness of the substrate is preferably from 100 μm to 1,000 μm inclusive. More preferably, the thickness is from 300 μm to 500 μm inclusive.

The substrate preferably has an adhesive layer on a portion on which the microneedles on the attachment surface that is a surface of the substrate on the microneedle side are not formed. This allows a skin and the microneedle device of the present disclosure to adhere to each other.

The microneedle device of the present disclosure may subcutaneously administer a medicine and withdraw a reaction product with the same microneedles, and the microneedles that subcutaneously administer a medicine and the microneedles that withdraw a reaction product may be different.

That is, the number of types of microneedles may be one or two or more.

Hereinafter, each embodiment of the microneedle device of the present disclosure will be described.

In each embodiment of the microneedle device of the present disclosure, since the configuration of the reaction product use portion is not particularly limited, illustration and description of the configuration of the reaction product use portion are omitted in the drawings and the description of each embodiment.

First, an example of an embodiment in which the number of types of microneedles is one will be described.

### First Embodiment

In a microneedle device according to a first embodiment of the present disclosure, the microneedle includes a needle substrate that is a porous material, and a medicine layer containing the medicine that is provided on the surface of the needle substrate, and the medicine contained in the medicine layer is subcutaneously administered, and the reaction product is withdrawn from the needle substrate.

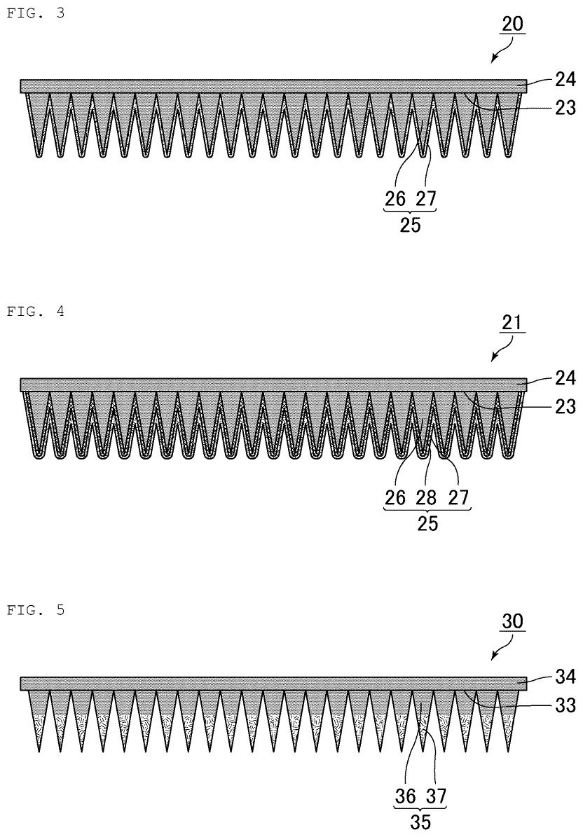

FIG. 3 is a sectional view schematically illustrating an example of the embodiment of a microneedle device.

In a microneedle device 20 illustrated in FIG. 3, microneedles 25 are provided on an attachment surface 23 of a substrate 24.

The microneedle 25 includes a needle substrate 26 that is a porous material and a medicine layer 27 provided on the surface of the needle substrate 26. When the needle substrate is a porous material, a reaction product can be withdrawn from the surface of the needle substrate.

As the needle substrate that is a porous material, the configuration of the porous microneedle described as the general configuration of the microneedle can be applied.

Also, as the material of the needle substrate, the material described as the general configuration of the microneedle can be used, but it is preferable to use a material having lower biodegradability than the medicine layer, and it is preferable that the needle substrate is not decomposed at the stage of administration of the medicine by decomposition of the medicine layer. Preferred examples of the material of the needle substrate include biodegradable biopolymers, and specific examples thereof include PLGA (poly(lactic-co-glycolic acid)), hyaluronic acid, and collagen.

The medicine layer 27 contains a medicine to be subcutaneously administered. The medicine layer may contain components other than the medicine, such as saccharides (lactose, raffinose, trehalose, sucrose, and the like), human albumin, polyglutamic acid, polyaspartic acid, polyhistidine, pentic acid, polylactic acid, hyaluronic acid, collagen, polyamino acid, hydroxypropylmethyl cellulose, hydroxypropylcellulose, dextran, polyvinyl alcohol, polyvinylpyrrolidone, polyethylene oxide, and chondroitin, in order to form a layer containing the medicine. Moreover, the entire medicine layer is preferably a material having high biodegradability.

The medicine layer preferably has a thickness of 1 μm to 100 μm inclusive.

When the microneedles 25 are inserted into a skin, a medicine is subcutaneously administered from the medicine layer 27. The medicine reacts in vivo to generate a reaction product.

Immediately after the attachment of the microneedles, the medicine is subcutaneously administered from the medicine layer. Since the surface of the needle substrate is covered with the medicine layer, a medicine administration function is exhibited prior to a reaction product withdrawal function by the microneedle.

When the medicine layer 27 is subcutaneously dispersed to expose the surface of the needle substrate 26 coated with the medicine layer 27, the reaction product withdrawal function by the needle substrate 26 that is a porous material is exhibited.

That is, the microneedle device of the present embodiment functions as a device that subcutaneously administers a medicine and withdraws a reaction product with a time difference.

In the microneedle device according to the first embodiment of the present disclosure, it is preferable that the surface of the needle substrate is coated with a hydrophilic material layer, the medicine layer is provided on the hydrophilic material layer, and the medicine is a hydrophobic material.

FIG. 4 is a sectional view schematically illustrating an example of the embodiment of a microneedle device.

A microneedle device 21 illustrated in FIG. 4 has substantially the same configuration as the microneedle device 20 illustrated in FIG. 3, but the surface of the needle substrate 26 is coated with a hydrophilic material layer 28, and the medicine layer 27 is provided on the hydrophilic material layer 28.

When the medicine layer 27 is subcutaneously dispersed by the attachment of the microneedles 25, the surface of the hydrophilic material layer 28 is exposed. When the surface of the needle substrate is coated with the hydrophilic material layer, an affinity with a hydrophilic reaction product is improved, so that the hydrophilic reaction product is easily withdrawn.

Since glycerol to be withdrawn as a reaction product in the above example is a hydrophilic substance, the withdrawing property of glycerol as a reaction product can be enhanced by providing the hydrophilic material layer.

That is, when the reaction product to be withdrawn is a hydrophilic substance, the withdrawing property of the reaction product can be enhanced by providing the hydrophilic material layer.

The hydrophilic material layer may be a layer composed only of a hydrophilic material or may contain a component that is not a hydrophilic material.

When the hydrophilic material layer is provided, the medicine is preferably a hydrophobic material. When the medicine is a hydrophobic material, an affinity with the surface of the hydrophilic material layer is low. Therefore, the administered medicine is inhibited from being withdrawn by the microneedle, and the medicine is easily moved to the living body side.

A lipase or an analog thereof as a lipolytic enzyme is a hydrophobic material.

Regarding the hydrophobicity and hydrophilicity of the materials in the present specification, protein and peptide can be discriminated by calculating Hydropathy from the amino acid sequence. It can also be determined by the solubility in water. Lipase may be discriminated by the value of the grand average of hydropathy (GRAVY).

The present specification describes an disclosure of a microneedle device including a substrate and microneedles provided on an attachment surface on the surface of the substrate, in which the microneedle includes a needle substrate that is a porous material, and a medicine layer containing the medicine that is provided on a surface of the needle substrate.

In addition, the present specification describes an disclosure of a medicine administration/reaction product withdrawal method including subcutaneously administering the medicine contained in the medicine layer by attaching the attachment surface including the microneedles to a living body and withdrawing a reaction product generated by subcutaneously administering the medicine from the needle substrate using the microneedle device.

### Second Embodiment

In a microneedle device according to a second embodiment of the present disclosure, the microneedle has a tip portion and a root portion, the tip portion contains the medicine, and the reaction product is withdrawn from the root portion.

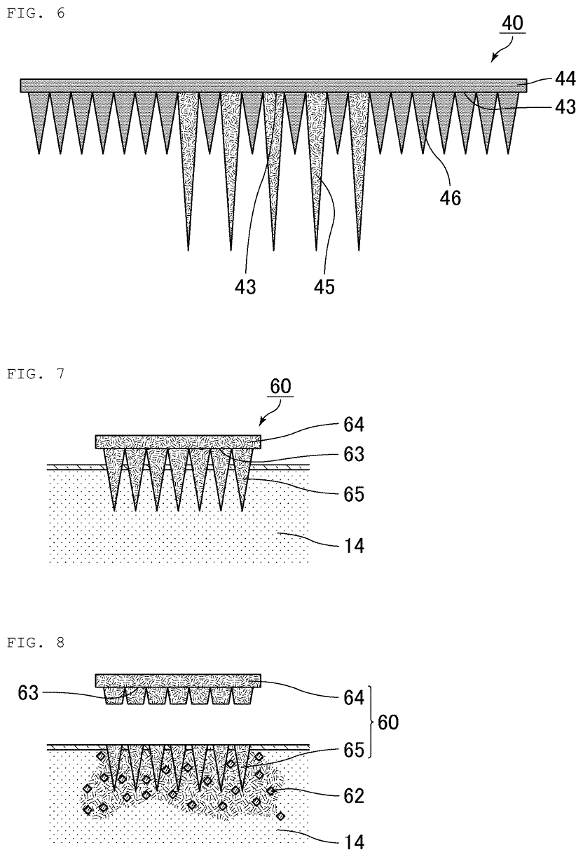

FIG. 5 is a sectional view schematically illustrating an example of the embodiment of a microneedle device.

In a microneedle device 30 illustrated in FIG. 5, microneedles 35 are provided on an attachment surface 33 of a substrate 34.

The microneedle 35 is divided into a tip portion 37 and a root portion 36. The tip portion 37 contains a medicine, and when the microneedles 35 are inserted into the skin, the medicine is subcutaneously administered from the tip portion 37. The medicine reacts in vivo to generate a reaction product.

The tip portion 37 is preferably made of a material that is soluble in the living body, and it is preferable that the tip portion dissolves to release the medicine.

The tip portion contains a medicine to be subcutaneously administered. The tip portion contains the medicine, and may contain a component that is biosoluble and moldable into the shape of the tip portion of the microneedle, such as saccharides (lactose, raffinose, trehalose, sucrose, and the like), human albumin, polyglutamic acid, polyaspartic acid, polyhistidine, pentic acid, polylactic acid, hyaluronic acid, collagen, polyamino acid, hydroxypropylmethyl cellulose, hydroxypropylcellulose, dextran, polyvinyl alcohol, polyvinylpyrrolidone, polyethylene oxide, and chondroitin, in order to form a soluble portion.

The root portion 36 is preferably a porous material.

As the material of the root portion that is a porous material, the material described as the general configuration of the microneedle can be used, but it is preferable to use a material having lower biodegradability than the tip portion, and it is preferable that the root portion is not decomposed at the stage of administration of the medicine from the tip portion.

As the material of the root portion, the same material as the needle substrate of the first embodiment of the microneedle device can be suitably used.

When the tip portion 37 dissolves in the living body, the medicine is subcutaneously dispersed and a reaction product is generated. Then, the reaction product withdrawal function by the root portion 36 is exhibited.

That is, the microneedle device of the present embodiment functions as a device that subcutaneously administers a medicine and withdraws a reaction product with a time difference.

The ratio between the root portion and the tip portion in the microneedle is not particularly limited.

The microneedle having the tip portion and the root portion can be manufactured such that the tip portion is extended to the tip of the root portion by dip coating the tip of the root portion after forming the root portion.

The present specification describes an disclosure of a microneedle device including a substrate and microneedles provided on an attachment surface on a surface of the substrate, in which the microneedle has a tip portion and a root portion, the tip portion contains a medicine, and the root portion withdraws a reaction product generated by subcutaneously administering the medicine.

In addition, the present specification describes an disclosure of a medicine administration/reaction product withdrawal method including subcutaneously administering the medicine contained in the tip portion by attaching the attachment surface including the microneedles to a living body and withdrawing a reaction product generated by subcutaneously administering the medicine from the root portion using the microneedle device.

Subsequently, an example of an embodiment in which the number of types of microneedles is two will be described.

In the following example, the microneedles include first microneedles and second microneedles, the first microneedle is a microneedle for subcutaneously administering the medicine, and the second microneedle is a microneedle for withdrawing the reaction product.

In the following description, the microneedle for administering the medicine is a first microneedle, and the microneedle for withdrawing the reaction product is a second microneedle.

The first microneedle may be a microneedle in which the microneedle itself dissolves to release the medicine.

In this case, the first microneedle contains the medicine, and may contain a component that is biosoluble and moldable into the shape of the first microneedle, such as saccharides (lactose, raffinose, trehalose, sucrose, and the like), human albumin, polyglutamic acid, polyaspartic acid, polyhistidine, pentic acid, polylactic acid, hyaluronic acid, collagen, polyamino acid, hydroxypropylmethyl cellulose, hydroxypropylcellulose, dextran, polyvinyl alcohol, polyvinylpyrrolidone, polyethylene oxide, and chondroitin, in order to form a soluble portion.

In addition, the first microneedle may be one in which the medicine is carried on the needle substrate, or may include a medicine layer containing the medicine provided on the surface of the needle substrate.

As the second microneedle, it is preferable to apply the configuration of the porous microneedle described as the general configuration of the microneedle. Also, the second microneedle may be a soluble material.

### Third Embodiment

In a microneedle device according to a third embodiment of the present disclosure, a length of the first microneedle is longer than a length of the second microneedle.

FIG. 6 is a sectional view schematically illustrating an example of the embodiment of a microneedle device.

In a microneedle device 40 illustrated in FIG. 6, first microneedles 45 and second microneedles 46 are provided on an attachment surface 43 of a substrate 44.

The first microneedle 45 is longer than the second microneedle 46.

Also, the ratio of the number of the first microneedles to the number of the second microneedles is not particularly limited.

Furthermore, the thickness of the first microneedle 45 is preferably smaller than the thickness of the second microneedle 46.

The thickness of the first microneedle and the thickness of the second microneedle are compared by a diameter of a bottom of the microneedle (the maximum diameter of the microneedle).

In addition, with respect to an aspect ratio which is a ratio of the length of the microneedle to the thickness of the microneedle (length of microneedle/thickness of microneedle (diameter of bottom)), the aspect ratio of the first microneedle 45 is preferably larger than the aspect ratio of the second microneedle 46.

The positions at which the first microneedles and the second microneedles are provided on the attachment surface are also not particularly limited, but it is preferable that the first microneedles are present at a center of the attachment surface and the second microneedles are present around the first microneedles at the center (peripheral portion of the attachment surface) in a plan view (bottom view) of the microneedle device from the microneedle side.

In addition, it is preferable that both the first microneedles and the second microneedles are present in the center of the attachment surface and the second microneedles are present in the peripheral portion of the attachment surface.

The first microneedle is a microneedle for subcutaneously administering a medicine, and when the length of the first microneedle is long, the medicine can be reliably administered to a deep region under the skin.

Since the second microneedle only needs to be able to withdraw the reaction product generated by reacting with the medicine and diffusing to the vicinity of the epidermis, it is not necessary to invade the second microneedle to a deep region under the skin. In addition, when the second microneedle is short, dissolution is slow, a through-hole can be secured in the epidermis for a long time, and the reaction product can be withdrawn for a long time.

That is, the length of the first microneedle used for administration of the medicine and the length of the second microneedle used for withdrawal of the reaction product are made different depending on the difference in roles.

A reaction product is generated by administering the medicine from the first microneedles 45 to a deep region under the skin. Then, the reaction product withdrawal function by the second microneedle 46 is exhibited.

That is, the microneedle device of the present embodiment functions as a device that subcutaneously administers a medicine and withdraws a reaction product with a time difference.

The present specification describes an disclosure of a microneedle device including a substrate and microneedles provided on an attachment surface of a surface of the substrate, in which the microneedles include first microneedles and second microneedles, the first microneedle is a microneedle for subcutaneously administering the medicine, the second microneedle is a microneedle for withdrawing the reaction product, and a length of the first microneedle is longer than a length of the second microneedle.

In addition, the present specification describes an disclosure of a medicine administration/reaction product withdrawal method including subcutaneously administering the medicine from the first microneedle by attaching the attachment surface to a living body and withdrawing from the second microneedle a reaction product generated by subcutaneously administering the medicine using the microneedle device.

### Fourth Embodiment

A microneedle device according to a fourth embodiment of the present disclosure includes a first device that includes the first microneedles and is peelable after releasing the medicine and a second device that includes the second microneedles and can withdraw the reaction product by piercing the second microneedles at a place where the first device has been peeled off.

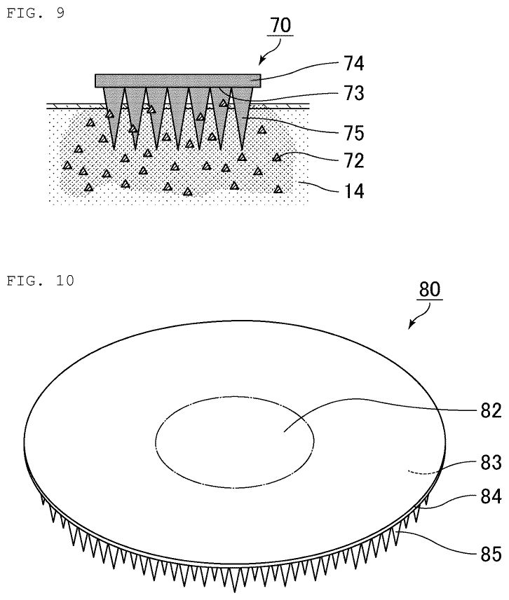

FIGS. 7, 8, and 9 are sectional views schematically illustrating an example of a procedure for performing administration of a medicine and withdrawal of a reaction product in the fourth embodiment of the microneedle device.

FIG. 7 illustrates a state in which a first device 60 is attached to a living body (skin tissue 14). The first device 60 includes a substrate 64 and first microneedles 65 provided on an attachment surface 63 of the substrate 64.

FIG. 8 illustrates a state in which a medicine 62 is released from the first microneedles 65 into the skin tissue 14 and the substrate 64 is peeled off. The first microneedle 65 is a biosoluble material and can be separated from the substrate 64.

When a predetermined time elapses after the substrate 64 has been peeled off, the medicine 62 released from the first microneedles 65 is accumulated subcutaneously, the reaction proceeds, and the first microneedles 65 dissolve and disappear.

In addition, when the first microneedle is not a biosoluble material, the first device is attached to the living body and left for a predetermined time, the medicine is released from the first microneedles, and then both the first microneedles and the substrate, that is, the entire first device is peeled off.

FIG. 9 illustrates a state in which a second device 70 is attached to a living body. The second device 70 includes a substrate 74 and second microneedles 75 provided on an attachment surface 73 of the substrate 74.

The second device 70 attaches to a position where the first device 60 has been peeled off.

The medicine is administered in the step shown in FIG. 8, whereby a reaction product 72 is generated and accumulated subcutaneously. The second device 70 is attached, and the subcutaneously accumulated reaction product 72 can be withdrawn by the second microneedles 75.

The medicine is administered subcutaneously by attachment of a first device including first microneedles to generate a reaction product. After the first device has been peeled off, the reaction product can be withdrawn by attachment of a second device including second microneedles.

That is, the microneedle device of the present embodiment functions as a device that subcutaneously administers a medicine and withdraws a reaction product with a time difference.

The present specification describes an disclosure of a microneedle device including a first device including a substrate and first microneedles for subcutaneously administering a medicine, and
a second device including a substrate and second microneedles for withdrawing a reaction product.

In addition, the present specification describes an disclosure of a medicine administration/reaction product withdrawal method including subcutaneously administering a medicine from the first microneedles by attaching the first device including the first microneedles to a living body, peeling the first device, and withdrawing a reaction product generated by subcutaneously administering the medicine by attaching the second device including the second microneedles to a position where the first device has been peeled off in the living body using the microneedle device.

### Fifth Embodiment

A microneedle device according to a fifth embodiment of the present disclosure includes a third device that includes the first microneedles on the attachment surface and has a cuttable peeling portion at a center of the attachment surface and a fourth device that includes the second microneedles and can withdraw the reaction product by being inserted into a space generated by cutting out the peeling portion to pierce the second microneedles.

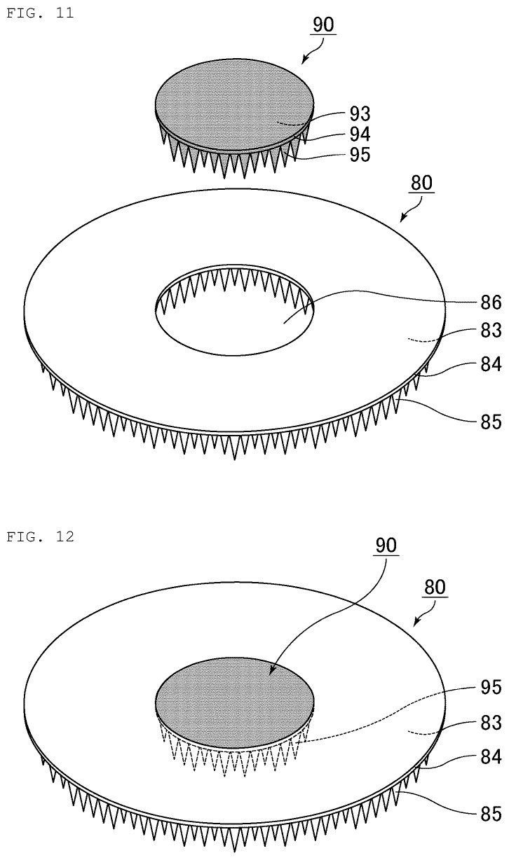

FIGS. 10, 11, and 12 are perspective views schematically illustrating an example of a procedure for performing administration of a medicine and withdrawal of a reaction product in the fifth embodiment of the microneedle device. FIGS. 10, 11, and 12 illustrate shapes viewed from a surface opposite to the attachment surface.

A third device 80 includes a substrate 84 and first microneedles 85 provided on an attachment surface 83 of the substrate 84. The first microneedles 85 are provided over the entire attachment surface 83 from the center to the periphery of the attachment surface 83.

The third device 80 has a cuttable peeling portion 82 at the center of the attachment surface 83 of the third device 80. In FIG. 10, the range of the peeling portion is indicated by a range of a circle surrounded by a one-dot chain line.

Although the third device 80 is drawn as having a circular plan view shape, the plan view shape is not limited to a circular shape, and may be another shape such as a polygon or an ellipse.

When the third device 80 is attached to the living body, the medicine is released from the first microneedles 85.

FIG. 11 illustrates a state in which a space has been generated by cutting out the peeling portion.

When the third device 80 is attached to the living body and left for a predetermined time, the medicine released from the first microneedles 85 is accumulated subcutaneously, and the reaction proceeds. After a predetermined time has elapsed, the peeling portion 82 is cut out to expose a space 86.

FIG. 11 also illustrates a fourth device 90. The fourth device 90 includes a substrate 94 and second microneedles 95 provided on an attachment surface 93 of the substrate 94.

The fourth device 90 is inserted into the space 86 obtained by cutting out the peeling portion 82 of the third device 80. The shape of the fourth device 90 is designed to match the shape of the space 86.

FIG. 12 illustrates a state in which the fourth device is inserted.

As illustrated in FIG. 12, the fourth device 90 is inserted into the space 86 to form a microneedle device in which the second microneedles 95 are located at the center of the attachment surface, and the first microneedles 85 are located around the attachment surface.

At the time of insertion of the fourth device 90, the medicine has already accumulated subcutaneously and the reaction product has been generated, so that the reaction product can be withdrawn by the second microneedles 95.

That is, the microneedle device of the present embodiment functions as a device that subcutaneously administers a medicine and withdraws a reaction product with a time difference.

In addition, the reaction product use portion may be integrated with the fourth device, and the reaction product withdrawn by the second microneedles can be immediately used in the reaction product use portion.

The present specification describes an disclosure of a microneedle device including a third device that includes a substrate, and first microneedles for subcutaneously administering a medicine that are provided on an attachment surface of a surface of the substrate and has a cuttable peeling portion at a center of the attachment surface, and

a fourth device that includes a substrate and second microneedles for withdrawing a reaction product and can withdraw the reaction product by being inserted into a space generated by cutting out the peeling portion to pierce the second microneedles.

In addition, the present specification describes an disclosure of a medicine administration/reaction product withdrawal method including subcutaneously administering a medicine from the first microneedles by attaching the third device including the first microneedles to a living body, cutting out the peeling portion to expose a space, and inserting the fourth device including the second microneedles into the space, thereby withdrawing a reaction product generated by subcutaneously administering the medicine using the microneedle device.

### Sixth Embodiment

In a microneedle device according to a sixth embodiment of the present disclosure, the second microneedles are provided in a central portion of the attachment surface, the first microneedles are provided in a peripheral portion, and a water absorption layer is provided on a surface opposite to the attachment surface at a position of a central portion of the substrate.

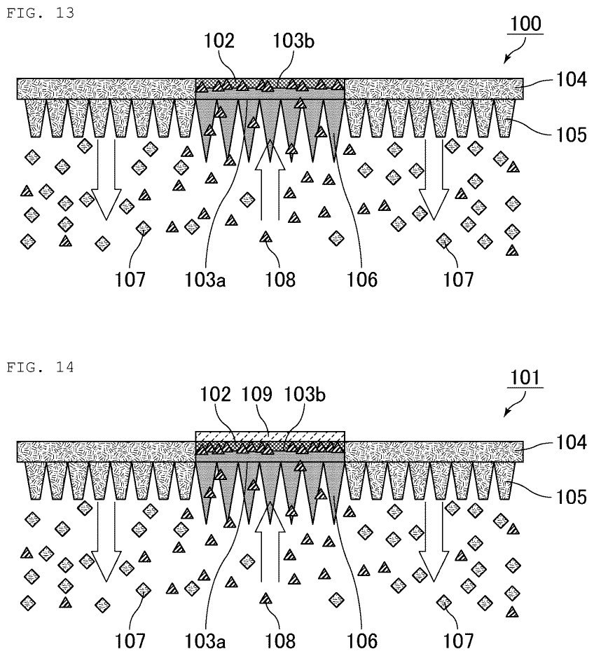

FIG. 13 is a sectional view schematically illustrating an example of the embodiment of a microneedle device.

In a microneedle device 100 illustrated in FIG. 13, second microneedles 106 are provided in a central portion of an attachment surface 103a of a substrate 104, and first microneedles 105 are provided in a peripheral portion of the attachment surface 103a of the substrate 104.

When the attachment surface 103a including the first microneedles 105 and the second microneedles 106 is attached to a living body, a medicine 107 is subcutaneously administered from the first microneedles 105. The medicine 107 is administered from the first microneedles 105 from the peripheral portion of the attachment surface 103a. The medicine 107 administered from the peripheral portion of the attachment surface 103a reacts subcutaneously to generate a reaction product 108.

In addition, the medicine 107 administered from the peripheral portion of the attachment surface 103a diffuses and reaches immediately below the central portion of the attachment surface 103a, and the reaction product 108 is also generated there.

Further, the reaction product 108 generated immediately below the peripheral portion of the attachment surface 103a diffuses and reaches immediately below the central portion of the attachment surface 103a.

The reaction product 108 accumulated immediately below the central portion of the attachment surface 103a is withdrawn by the second microneedles 106.

In FIG. 13, the administration direction of the medicine and the withdrawal direction of the reaction product are indicated by arrows.

The medicine 107 is administered from the first microneedles 105 to the peripheral portion of the attachment surface 103a to generate the reaction product 108, and the reaction product 108 is withdrawn by the second microneedles 106 in the central portion of the attachment surface 103a.

That is, the microneedle device of the present embodiment functions as a device that subcutaneously administers a medicine and withdraws a reaction product with a time difference.

A water absorption layer 102 is provided on a surface 103b opposite to the attachment surface of the substrate. The water absorption layer 102 stores the reaction product 108 withdrawn by the second microneedles 106 and concentrates the reaction product 108.

The water absorption layer 102 is detachable from the microneedle device, and the reaction product 108 concentrated in the water absorption layer 102 can be used in the reaction product use portion by mounting the water absorption layer 102 on the reaction product use portion (not illustrated).

The thickness and size of the water absorption layer are not particularly limited.

In addition, an evaporation promoting layer is preferably provided outside the water absorption layer.

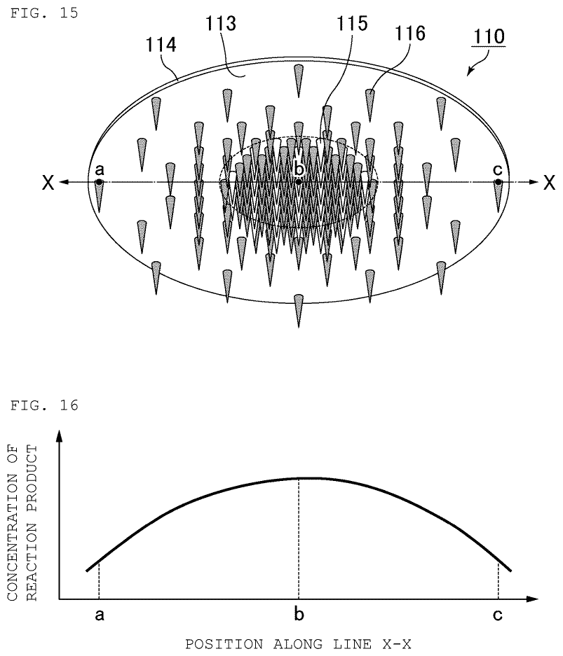

FIG. 14 is a sectional view schematically illustrating an example of the embodiment of a microneedle device.

In a microneedle device 101 illustrated in FIG. 14, an evaporation promoting layer 109 is provided outside the water absorption layer 102.

When the evaporation promoting layer is provided outside the water absorption layer, water is absorbed by the evaporation promoting layer from the components accumulated in the water absorption layer, and is evaporated and removed from the evaporation promoting layer. As a result, the concentration of the reaction product accumulated in the water absorption layer can be further increased.

Examples of the material of the evaporation promoting layer include graphene. The thickness and size of the evaporation promoting layer are not particularly limited.

The present specification describes an disclosure of a microneedle device including a substrate, second microneedles provided in a central portion of an attachment surface on a surface of the substrate, first microneedles provided in a peripheral portion of the attachment surface of the surface of the substrate, and a water absorption layer provided on a surface opposite to the attachment surface at a position of the central portion of the substrate.

In addition, the present specification describes an disclosure of a medicine administration/reaction product withdrawal method including subcutaneously administering the medicine from the first microneedle by attaching the attachment surface to a living body, withdrawing a reaction product generated by subcutaneously administering the medicine from the second microneedle, and further accumulating the reaction product in a water absorption layer using the microneedle device.

### Seventh Embodiment

In a microneedle device according to a seventh embodiment of the present disclosure, the first microneedles are provided in a central portion of the attachment surface, the second microneedles are provided over the entire attachment surface, and a density of the second microneedles on the attachment surface is high in the central portion and low in the peripheral portion.

FIG. 15 is a perspective view schematically illustrating an example of the embodiment of a microneedle device.

FIG. 15 illustrates a shape of the microneedle device as viewed from the attachment surface.

FIG. 16 is a graph schematically showing a concentration distribution of a reaction product along an axis indicated by line X-X in FIG. 15. In the horizontal axis of the graph of FIG. 16, left point a in FIG. 15 corresponds to the left side of the horizontal axis, center point b corresponds to the center of the horizontal axis, and right point c corresponds to the right side of the horizontal axis.

In a microneedle device 110 illustrated in FIG. 15, first microneedles 115 are provided in a central portion of an attachment surface 113 of a substrate 114, and second microneedles 116 are provided over the entire attachment surface 113 of the substrate 114.

When the attachment surface 113 including the first microneedles 115 and the second microneedles 116 is attached to a living body, a medicine is subcutaneously administered from the first microneedles 115. The medicine is administered from the first microneedles 115 from the central portion of the attachment surface 113. The medicine administered from the central portion of the attachment surface 113 reacts subcutaneously to generate a reaction product. The concentration of the reaction product is high immediately below the central portion of the attachment surface (point b on the X-X axis) and low immediately below the peripheral portion of the attachment surface (point a and point c on the X-X axis). FIG. 16 shows the concentration distribution of the reaction product when the reaction product is generated.

Since it is preferable to withdraw more reaction product in the central portion of the attachment surface 113 where the concentration of the reaction product is relatively high, the density of the second microneedles 116 for withdrawing a reaction product is increased in the central portion of the attachment surface 113. Since the concentration of the reaction product is low in the peripheral portion of the attachment surface 113, the density of the second microneedles 116 is low.

By determining the arrangement and density of the first microneedles and the second microneedles in this manner, the reaction product can be efficiently withdrawn.

In addition, the density of the second microneedles may gradually decrease from the central portion to the peripheral portion.

Also in the microneedle device of the present embodiment, since the subcutaneous administration of a medicine from the first microneedles is performed first and then the withdrawal of a reaction product by the second microneedles is performed, the microneedle device of the present embodiment functions as a device that subcutaneously administers a medicine and withdraws a reaction product with a time difference.

The present specification describes an disclosure of a microneedle device including a substrate, first microneedles provided in a central portion of an attachment surface on a surface of the substrate, and second microneedles provided in a peripheral portion of the attachment surface of the surface of the substrate, in which a density of the second microneedles on the attachment surface is high in the central portion and low in the peripheral portion.

In addition, the present specification describes an disclosure of a medicine administration/reaction product withdrawal method including subcutaneously administering the medicine from the first microneedle by attaching the attachment surface to a living body and withdrawing from the second microneedle a reaction product generated by subcutaneously administering the medicine using the microneedle device.

### [Medicine Administration/Reaction Product Withdrawal Method]

A medicine administration/reaction product withdrawal method of the present disclosure is a medicine administration/reaction product withdrawal method including preparing a microneedle device including a substrate and microneedles provided on an attachment surface on a surface of the substrate, subcutaneously administering a medicine by attaching the attachment surface including the microneedles to a living body, and withdrawing a reaction product generated by subcutaneously administering the medicine.

By using the microneedle device of the present disclosure described herein, it is possible to perform the medicine administration/reaction product withdrawal method of the present disclosure including administering a medicine and withdrawing a reaction product.

### DESCRIPTION OF REFERENCE SYMBOLS

1: Microneedle device
2: Reaction product use portion (battery unit)
3: Attachment surface
4: Substrate
5: Microneedle
6: Anode
7: Cathode
8: Separator
9: External circuit
10: Lipolytic enzyme
11: Glycerol oxidase
12: Oxygen reductase
13: Glycerol
14: Skin tissue
15: Adipocyte
20, 21: Microneedle device
23: Attachment surface
24: Substrate
25: Microneedle
26: Needle substrate
27: Medicine layer
28: Hydrophilic material layer
30: Microneedle device
33: Attachment surface
34: Substrate
35: Microneedle
36: Root portion
37: Tip portion
40: Microneedle device
43: Attachment surface
44: Substrate
45: First microneedle
46: Second microneedle
50: Microneedle device
53: Attachment surface
54: Substrate
55: First microneedle
56: Second microneedle
60: First device
62: Medicine
63: Attachment surface
64: Substrate
65: First microneedle
70: Second device
72: Reaction product
73: Attachment surface
74: Substrate
75: Second microneedle
80: Third device
82: Peeling portion
83: Attachment surface
84: Substrate
85: First microneedle
86: Space generated by cutting out peeling portion
90: Fourth device
93: Attachment surface
94: Substrate
95: Second microneedle
100, 101: Microneedle device
102: Water absorption layer
103a: Attachment surface of substrate
103b: Surface opposite to attachment surface of substrate
104: Substrate
105: First microneedle
106: Second microneedle
107: Medicine
108: Reaction product
109: Evaporation promoting layer
110: Microneedle device
113: Attachment surface
114: Substrate
115: First microneedle
116: Second microneedle

1. A microneedle device comprising:
a substrate; and
microneedles on an attachment surface on a surface of the substrate, wherein
the microneedle device constructed to subcutaneously administer a medicine when the attachment surface including the microneedles is attached to a living body, and to withdraw a reaction product generated by the subcutaneous administration of the medicine,
the medicine is a lipolytic enzyme or a peptide compound having an activity equivalent thereto, and
the reaction product is glycerol.

2. The microneedle device according to claim 1, wherein the microneedle has a tip portion and a root portion, the tip portion contains the medicine, and the reaction product is withdrawn from the root portion.

3. The microneedle device according to claim 2, wherein the tip portion is constructed to dissolve so as to release the medicine.

4. The microneedle device according to claim 2, wherein the root portion is a porous material.

5. A microneedle device comprising:
a substrate; and
microneedles on an attachment surface on a surface of the substrate, wherein
the microneedle device constructed to subcutaneously administer a medicine when the attachment surface including the microneedles is attached to a living body, and to withdraw a reaction product generated by the subcutaneous administration of the medicine,
the microneedle includes a needle substrate that is a porous material, and a medicine layer containing the medicine on a surface of the needle substrate, and
the reaction product is withdrawn from the needle substrate.

6. The microneedle device according to claim 5, the porous body has a D50 pore diameter distribution of 0.05 μm to 10 μm.

7. The microneedle device according to claim 5, further comprising a hydrophilic material layer on the surface of the needle substrate, the medicine layer is on the hydrophilic material layer, and the medicine is a hydrophobic material.

8. The microneedle device of claim 5, wherein
the medicine is a lipolytic enzyme or a peptide compound having an activity equivalent thereto, and
the reaction product is glycerol.

9. A microneedle device comprising:
a substrate; and
microneedles on an attachment surface on a surface of the substrate, wherein
the microneedle device constructed to subcutaneously administer a medicine when the attachment surface including the microneedles is attached to a living body, and to withdraw a reaction product generated by the subcutaneous administration of the medicine,
the microneedles include first microneedles and second microneedles, and
the first microneedles are constructed for the subcutaneously administering of the medicine, and the second microneedle constructed for the withdrawing of the reaction product.

10. The microneedle device according to claim 9, wherein the first microneedle is constructed to dissolve to release the medicine.

11. The microneedle device according to claim 9, wherein a length of the first microneedle is longer than a length of the second microneedle.

12. The microneedle device according to claim 9, wherein a thickness of the first microneedle is thinner than a thickness of the second microneedle.

13. The microneedle device according to claim 9, further comprising:
a first device that includes the first microneedles and is peelable after releasing the medicine; and
a second device that includes the second microneedles and constructed to withdraw the reaction product by piercing the second microneedles at a place where the first device has been peeled.

14. The microneedle device according to claim 9, further comprising:
a third device that includes the first microneedles on the attachment surface and has a cuttable peeling portion at a center of the attachment surface; and
a fourth device that includes the second microneedles and constructed to withdraw the reaction product by being inserted into a space generated by the cutting of the peeling portion to pierce the second microneedles.

15. The microneedle device according to claim 9, wherein
the second microneedles are in a central portion of the attachment surface, the first microneedles are in a peripheral portion of the attachment surface, and the microneedle device further comprises:
a water absorption layer on a surface of the substrate opposite to the attachment surface and at a position corresponding to a central portion of the substrate.

16. The microneedle device according to claim 15, further comprising an evaporation promoting layer outside of the water absorption layer.

17. The microneedle device according to claim 9, wherein
the first microneedles are in a central portion of the attachment surface, the second microneedles are located over an entirety of the attachment surface, and
a density of the second microneedles on the attachment surface is higher in the central portion and compared to the second microneedles in the peripheral portion.

18. The microneedle device of claim 9, wherein
the medicine is a lipolytic enzyme or a peptide compound having an activity equivalent thereto, and
the reaction product is glycerol.
